(19) United States
(12) Patent Application Publication
(10) Pub. No.: US 2024/0299241 A1
(43) Pub. Date: Sep. 12, 2024

(54) VIBRATORY DEVICE AND METHOD FOR USE OF SAME

(71) Applicant: Craig A. Hoffman, Austin, TX (US)

(72) Inventor: Craig A. Hoffman, Austin, TX (US)

(21) Appl. No.: 18/476,501

(22) Filed: Sep. 28, 2023

Related U.S. Application Data
(63) Continuation-in-part of application No. 18/118,058, filed on Mar. 6, 2023, now Pat. No. 11,771,953.

(51) Int. Cl. A61H 19/00; A61H 23/02

(52) U.S. Cl. CPC A61H 19/44; A61H 23/0263; A61H 2201/1215

(57) ABSTRACT

A vibratory device and method for its use are disclosed. It features an elongated body suitable for insertion into a pelvic cavity. The device comprises a haptic actuator, interconnectedith a processor, which generates a variety of vibrations through actuatable elements embedded within the device. The haptic actuator's unique design allows it to separate vibration amplitude from frequency in response to a haptic signal. Additionally, a manual actuator produces a user-input signal upon interaction. The device's processor may create a haptic signal based on this user-input signal. The design offers diverse application possibilities, including a range of haptic actuator variations from a magnetic wideband vibrational actuator to a solenoid actuator vibrator, a linear resonant actuator vibration motor, or a Linear Magnetic Ram (LMR) actuator. This vibratory device provides an immersive interactive experience, aiding users in enhancing their sexual experiences and understanding their bodies better.

### PRIORITY STATEMENT

This application is a continuation-in-part of U.S. patent application Ser. No. 18/118,058 entitled “Vibratory device and Method for Use of Same” and filed in the name of Craig A. Hoffman on Mar. 6, 2023, now U.S. Pat. No. 11,771,953 issued on Oct. 3, 2023; which is hereby incorporated by reference, in entirety, for all purposes.

### TECHNICAL FIELD OF THE INVENTION

This invention pertains to devices designed for enhancing sexual pleasure and wellness, specifically through the stimulation of the genital area and, in particular, this invention is particularly relevant to vibratory devices that can be used to stimulate and engage the muscles of the vulva, including the pubococcygeal and related perineal musculature, in females and male genital area.

### BACKGROUND OF THE INVENTION

A significant area of interest for individuals and health care providers alike is the enhancement of sexual pleasure and wellness. The sexual pelvic area, including the pelvic floor muscles of the pelvic area, play a crucial role in sexual satisfaction and overall genital health. The pelvic area muscles can be under-stimulated due to various factors such as age, lifestyle, or lack of use, leading to decreased sexual pleasure and responsiveness.

Various devices have been developed to stimulate and engage the sexual pelvic region, with the specific goal of enhancing sexual pleasure and responsiveness. Despite the existence of such devices, there is a continuing interest in an improved device that allows users to engage the sexual pelvic region in a comfortable and private setting. This device should be user-friendly, carry a low risk of injury, and be easy to maintain. There is also a growing demand for devices that provide a more immersive and interactive experience. This would aid in training users to better understand their bodies and enhance their sexual experiences. Such a device could be particularly beneficial for individuals experiencing decreased sexual pleasure or responsiveness due to under-stimulation of the sexual pelvic region.

### SUMMARY OF THE INVENTION

It would be advantageous to develop a vibratory device and method for its use that significantly enhances the current state of devices designed for sexual pleasure and wellness. It would be desirable to enable a mechanical and electronics-based solution that provides enhanced performance, improved usability, and an enriched feature set. It would be further desirable to enable a mechanical and electronics-based solution that provides an immersive and interactive experience for users, aiding them in enhancing their sexual experiences and understanding their bodies better. To better address one or more of these concerns, a vibratory device and method for its use are disclosed.

In one embodiment of the vibratory device, an elongated shaft with an external surface portion is designed for insertion within a pelvic cavity. A haptic actuator, having processing that separates amplitude from frequency, generates vibrations via actuatable elements integrated into the vibratory device in response to receiving a haptic signal from a processor. The haptic signal may be based on a manual signal from a manual actuator.

In another aspect, in one embodiment of the vibratory device, an elongated shaft houses actuatable elements. A haptic actuator, having processing that separates amplitude from frequency, generates vibrations, which may be a tapping sensation, via actuatable elements integrated into the vibratory device in response to receiving a haptic signal. Within the vibratory device, memory is accessible to the processor and includes processor-executable instructions that, when executed by a processor, cause the processor to receive the manual signal, create the haptic signal based on the manual signal, and drive the haptic signal to the haptic actuator. A frequency adjustment sensor may also be included. These and other aspects of the invention will be apparent from and elucidated with reference to the embodiments described hereinafter.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a simplified sectional view of the pelvic region of the female anatomy, showing one embodiment of a vibratory device inserted within the intravaginal cavity in the operative actuation position, according to the teachings presented herein;

FIG. 2 is a rear perspective of view depicting the vibratory device of FIG. 1A being utilized according to the teachings presented herein;

FIG. 3 is a front elevation view of the vibratory device depicted in FIG. 2;

FIG. 4 is a side elevation view, partly in section, of the vibratory device probe depicted in FIG. 2;

FIG. 5 is a sectional view of the vibratory device as taken along line 5-5 of FIG. 4;

FIG. 6 is a functional block diagram of one embodiment the vibratory device depicted in FIG. 1;

FIG. 7 is a conceptualized functional block diagram of another embodiment of the vibratory device probe according to the teachings presented herein; and

FIG. 8 is a conceptualized functional block diagram of a still further embodiment of the vibratory device probe according to the teachings presented herein;

### DETAILED DESCRIPTION OF THE INVENTION

While the making and using of various embodiments of the present invention are discussed in detail below, it should be appreciated that the present invention provides many applicable inventive concepts, which can be embodied in a wide variety of specific contexts. The specific embodiments discussed herein are merely illustrative of specific ways to make and use the invention, and do not delimit the scope of the present invention.

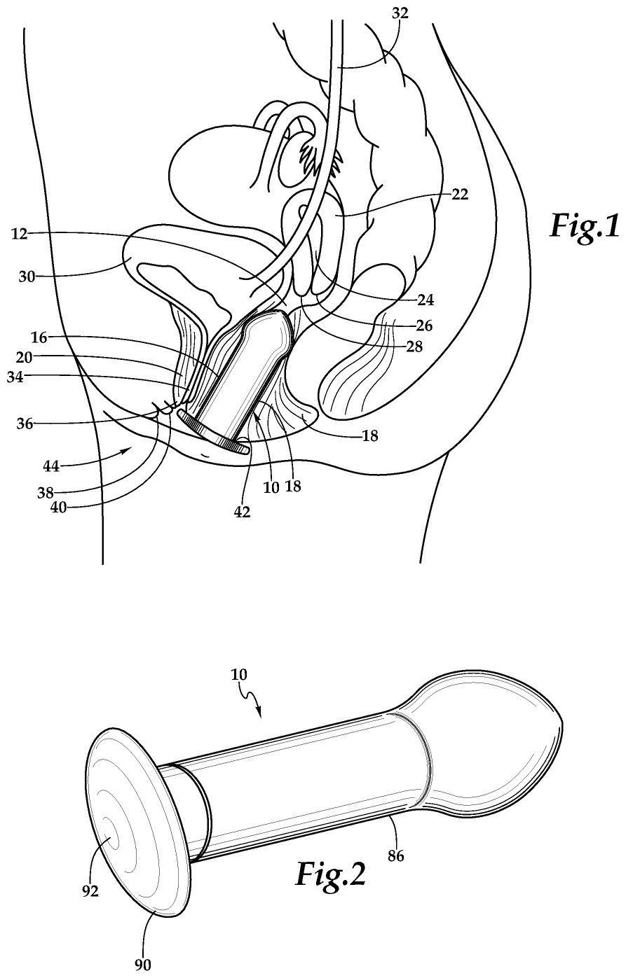

Referring initially to FIG. 1, therein is depicted one embodiment of a vibratory device for pleasure enhancement, which is schematically illustrated and designated 10. The vibratory device 10 presented herein may be inserted in the pelvic cavity 12 while the user is reclining in a comfortable position. In that position, the user is lying on their back, knees raised, with their head slightly elevated relative to the pelvic region. Their torso is on an approximate 30-degree angle with respect to horizontal, which results in a half-sitting position, which is a preferred position for using the device.

The lower wall 14 and the upper wall 16 of the vagina are connected to muscles, tissues, and nerves, that are indicated generally at 18, 20, and collectively referred to herein as the pubococcygeal and related perineal musculature. Also shown is the uterus 22, which has an internal void known as the uterine body cavity 24, as well as the cervix 26 and the external os 28, which is the external opening of the cervix facing the vaginal cavity 12. Other portions of the female anatomy shown in FIG. 1 include the bladder 30, ureter 32, the urethra 34, the labium minus 36, the labium majus 38, which join together in the region adjacent the clitoris 40, near the vaginal introitus 42, all of which are clustered about the region generally known as the perineum 44. In one embodiment of the vibratory device 10, the vibratory device 10 includes processing that separates amplitude from frequency when generating vibrations, which may be a tapping sensation, via actuatable elements integrated into the sleeve. Further, the vibratory device 10 senses external pressure forces by way of resistive or capacitive sensing and drives a feedback signal based on the sensed external pressure forces.

The vibratory device 10, as depicted in FIG. 1, is designed with a primary focus on enhancing pleasure for the user. The device's unique design and functionality aim to provide an immersive and interactive experience that goes beyond the capabilities of traditional pleasure enhancement devices. The vibratory device 10 is not merely a tool for physical stimulation but is also designed to engage the user on a deeper, more personal level, promoting a greater understanding of their body and its responses. The vibratory device 10 is designed to be inserted into the pelvic cavity 12, where it can interact directly with the sensitive tissues and muscles of the pubococcygeal and related perineal musculature. The device's elongated shaft is designed to comfortably fit within the user's body, providing stimulation directly to the areas that are most responsive to pleasurable sensations. The device's haptic actuator is capable of generating a wide range of vibrations, from gentle pulsations to more intense vibrations, allowing the user to customize their experience based on their personal preferences and comfort level.

The vibratory device 10 is also designed to provide a unique form of tactile feedback. The device's haptic actuator separates amplitude from frequency when generating vibrations, which can create a tapping sensation that many users find particularly pleasurable. This unique form of stimulation can provide a novel sensory experience that sets the vibratory device 10 apart from other pleasure enhancement devices. The vibratory device 10 also includes a sensor that senses external pressure forces and drives a feedback signal based on the sensed external pressure forces. This feedback signal can provide valuable information to the user about their body's responses, helping them to better understand their own pleasure responses and to adjust their use of the device accordingly. This feedback mechanism can also help to ensure that the device is being used safely and comfortably.

In addition to enhancing pleasure, the vibratory device 10 can also have other beneficial effects. For example, regular use of the device can help to promote blood flow to the pelvic region, which can have a range of health benefits. Increased blood flow can help to promote tissue health, improve sexual response, and even help to alleviate certain types of discomfort or pain. While the vibratory device 10 is primarily designed for use by individuals with a vagina, it is important to note that the device can also be used by individuals with a penis in other embodiments. The device's unique design and functionality make it versatile enough to provide pleasure enhancement for individuals of all genders. For example, the device could be used to provide stimulation to the prostate, a sensitive area that can be accessed through the rectum. This versatility further expands the potential user base for the vibratory device 10, making it a truly inclusive pleasure enhancement device. That is, the vibratory device 10 represents a significant advancement in the field of pleasure enhancement devices. Its unique design and functionality, combined with its focus on user feedback and understanding, set it apart from traditional devices. Whether used for enhancing pleasure, promoting health, or exploring one's body, the vibratory device 10 offers a unique and immersive experience that can benefit users of all genders.

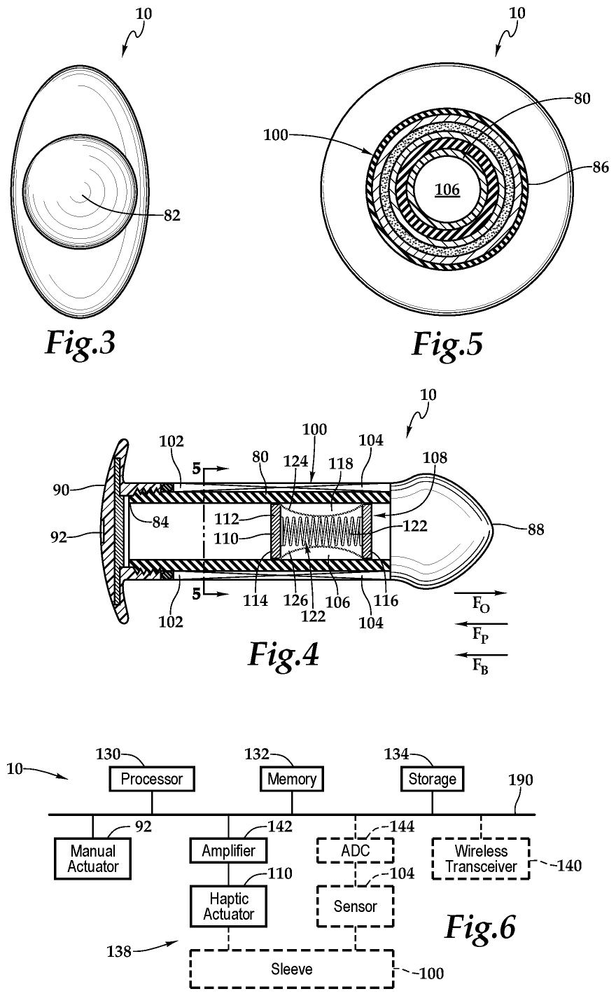

Referring now to FIG. 2 through FIG. 6, in some embodiments, the vibratory device 10 includes an elongated shaft 80 having an insertion end 82 and a base end 84 with an external surface portion 86 therebetween. The elongated shaft 80 is terminated at the insertion end 82 by a slightly enlarged and rounded head portion 88. As previously detailed in FIG. 1, the elongated shaft 80 is receivable within the pelvic cavity 12. A handle 90 is located at the base end 84. The handle 90 may also function as a closure cap. In such an embodiment, the handle 90 is fitted with threads engaging mating threads that are formed at the base end 84 of the elongated shaft 80. A manual actuator 92 is secured within the handle 90 and presented for use by a user at thereat. The manual actuator 92 may provide various manual control for regulating the applied vibration and have an ON/OFF switch integrated therewith. Additionally, in some embodiments, a display (not shown) may be positioned on the handle to provide information to a user.

In one embodiment, an optional sleeve 100 is positioned lengthwise and circumferentially about the elongated shaft 80 proximate the external surface portion 86. The sleeve 100 may have actuatable members 102 and an optional sensor 104 therein. The elongated shaft 80 surrounds a pocket portion 106. Within the pocket portion 106, electronic components 108, including a haptic actuator 110, are positioned. The haptic actuator 110 has processing that separates amplitude from frequency, and the haptic actuator 110, in response to receiving a haptic signal, generates vibrations, which may be a tapping sensation, via the actuatable elements 102, which may be integrated into the sleeve 100. The actuatable elements 102 may include magnetic field-based actuators with a moving magnet or a moving coil, or the like, for example. The sensor 104 may include an accelerometer, gyroscope, proximity sensor, inclinometer, or the like, for example. Various components may be at least partially integrated depending on the architecture and design of the vibratory device 10. By way of example, and not by way of limitation, the haptic actuator 110 and the sleeve 100 may be at least partially integrated. Similarly, the sensor 104 and the sleeve 100 may be at least partially integrated. Further, the haptic actuator 110 and the sleeve 100 may be at least partially integrated.

Returning to the haptic actuator 110, the haptic actuator 110 may be a magnetic wideband vibrational actuator, a solenoid actuator vibrator, or a linear resonant actuator vibration motor, or a Linear Magnetic Ram (LMR), for example. The haptic actuator 110 may include a moving mass as well as a sensing member that respond to the mass of the vibratory device and to the mass of the body. By way of example, in applications using a voice coil actuator, the moving mass may include one or more permanent magnets which are set into motion by inducing a magnetic field by applying a current to voice coil proximate the one or more permanent magnets. The force of the moving mass is opposed by both the vibratory device itself and the human body. Various reinforcement and dampening measures between the vibratory device 10 and the moving mass therein offset the force from the vibratory device 10.

In one embodiment, the haptic actuator 110 includes a magnet and a hollow member, including a coil therein, with relative longitudinal movement therebetween with thin elastic membranes interconnecting the magnet and the hollow member. In another embodiment, which is illustrated in FIG. 5, the haptic actuator 110 may include a magnet frame 112, including a magnets 114, 116 therein, and a hollow member 118, including a coil 120, which is shown as a voice coil 122, therein, that have relative longitudinal movement therebetween with thin elastic membranes 124, 126 interconnecting the magnets 114, 116 and the hollow member 118. In the embodiments with the magnet frame 112, the magnets 114, 116 within the magnet frame 112 may be within an arrangement with same polarities facing each other inside the magnet frame 112. As shown, the force F_{O} of the moving mass, the magnets 114, 116 within the magnet frame 112, is opposed by both the vibratory device F_{P} itself and the human body F_{B}. More generally, for interactive events, such as interfacing with a portion of the human body with the human body's geometry, contours, and texture, kinaesthetic, tactile, and proprioceptic information is integrated into haptics. This vibrotactile percept requires active participation by the user, the human body, and the vibratory device itself and all characteristics, from velocity to direction of movement have an influence on resulting haptic stimulus.

Returning to the haptic actuator 110, as mentioned, the haptic actuator 110 may be a magnetic wideband vibrational actuator, a solenoid actuator vibrator, a linear resonant actuator vibration motor, or a Linear Magnetic Ram (LMR), among others. The LMR is a wideband voice-coil haptic motor that uses a solid-state magnetic suspension instead of springs or flexures. This technology provides class-leading frequency response, reliability, and strength of haptic output. The LMR's solid-state magnetic suspension eliminates the need for mechanical springs, resulting in a more robust and durable haptic actuator. The wideband frequency response of the LMR allows it to generate a broad range of tactile sensations, from subtle vibrations to strong pulses, enhancing the user's sensory experience. The LMR's high reliability ensures consistent performance over time, providing users with a dependable and satisfying haptic experience. The strong haptic output of the LMR allows it to deliver powerful tactile sensations, enhancing the user's pleasure and satisfaction.

As mentioned, the elongated shaft 80 houses electronic components 108. More particularly, the elongated shaft 80 may house a processor 130, memory 132, and storage 134 communicatively interconnected in a busing architecture 136 within the pocket. The manual actuator 92 may also be connected to the busing architecture 136. Further, haptic elements 138 are communicatively interconnected to the busing architecture 136. The haptic elements 138 may include the haptic actuator 110 having an amplifier 142 driving a signal thereto. As shown, the haptic actuator 110 drives a signal to the sleeve 100. As part of the haptic elements 138, the sensor 104 measures physical properties, such as sensing external pressure forces, of the sleeve 100 and drives a signal to an optional analog-to-digital convertor 144, which is coupled to the busing architecture 136.

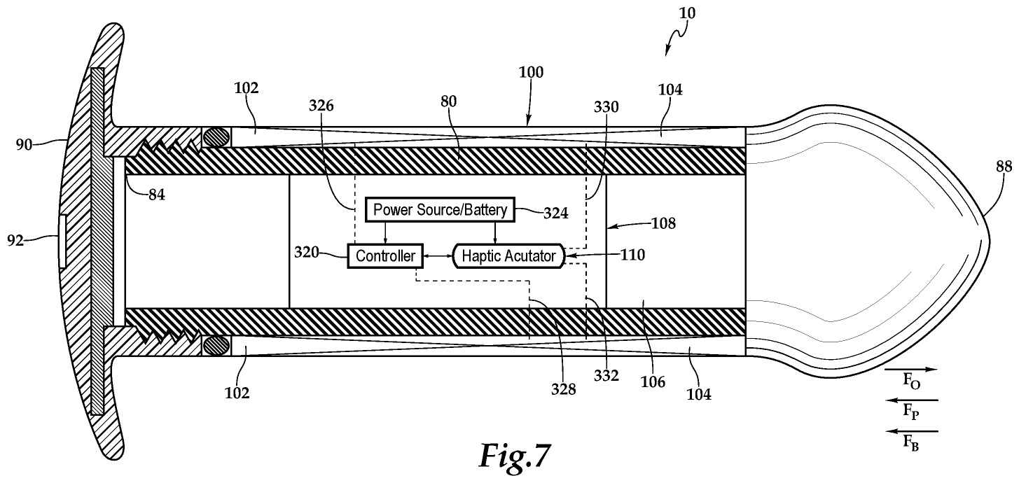

As shown, an optional wireless transceiver 140 may be a transmitter/receiver or an antenna, for example, that is coupled to the busing architecture 190. Communication between various smart devices and the vibratory device 10 may be enabled by a variety of wireless methodologies employed by the wireless transceiver 140, including 802.11, 3G, 4G, Edge, WiFi, ZigBee, near field communications (NFC), Bluetooth low energy, and Bluetooth, for example. The various controls and inputs and outputs presented above are exemplary and it should be appreciated that other types of controls may be incorporated in the vibratory device 10. Moreover, the electronics and form of the vibratory device 10 may vary. By way of example, and not by way of limitation, an alternate embodiment of the vibratory device 10 is presented in FIG. 7 hereinbelow.

The memory 132 and storage 134 are accessible to the processor 130 and include processor-executable instructions that, when executed, cause the processor 130 to execute a series of operations. In a first series of operations, the processor-executable instructions cause the processor 130 to receive the feedback signal, which is driven by the sensor 104 following the sensor 104 sensing external pressure forces. The processor is then caused to create the haptic signal based on a manual signal and the feedback signal. The manual signal is generated by the manual actuator 92 in response to manual actuation thereof. The haptic signal is then driven by the processor to the haptic actuator.

The memory 132 may further include processor-executable instructions that, when executed by the processor 130, cause the processor 130 to execute a manual tapping output mode. In one operational mode, the vibration frequency range of the haptic actuator 110 may be between 0 Hz to 80 Hz. In another operational mode, the vibration frequency range of the haptic actuator 110 may be between 0 Hz to 120 Hz.

The vibratory device 10, as depicted in FIGS. 2 through 6, is designed with user comfort and pleasure in mind. The elongated shaft 80, with its insertion end 82 and base end 84, is crafted to fit comfortably within the pelvic cavity 12. The slightly enlarged and rounded head portion 88 at the insertion end 82 is designed to enhance the user's pleasure by stimulating sensitive areas within the pelvic cavity. The external surface portion 86 of the elongated shaft 80 is smooth and designed to minimize discomfort during insertion and removal. The handle 90 at the base end 84 of the elongated shaft 80 provides the user with control over the device. It is designed to be easy to grip, allowing the user to adjust the position of the device as needed. The manual actuator 92, secured within the handle 90, provides the user with control over the device's vibrations. The user can turn the device on or off and adjust the intensity of the vibrations to suit their preferences. In some embodiments, a display may be positioned on the handle to provide information to the user, such as battery life or vibration settings.

The optional sleeve 100, positioned lengthwise and circumferentially about the elongated shaft 80, is designed to enhance the user's experience. The sleeve 100 may contain actuatable members 102 that generate vibrations in response to a haptic signal from the haptic actuator 110. These vibrations can range from a gentle hum to a powerful throb, depending on the user's preferences. This allows the device to adjust its vibrations in response to the user's movements, creating a more interactive and personalized experience.

The haptic actuator 110, positioned within the pocket portion 106 of the elongated shaft 80, is the heart of the vibratory device 10. It separates amplitude from frequency to create a wide range of vibrations, from a gentle tapping sensation to a powerful throb. The haptic actuator 110 can be a magnetic wideband vibrational actuator, a solenoid actuator vibrator, a linear resonant actuator vibration motor, or a Linear Magnetic Ram (LMR) actuator, among others. The choice of haptic actuator can be tailored to the user's preferences, allowing for a highly personalized experience. The electronic components 108 within the elongated shaft 80, including the processor 130, memory 132, and storage 134, allow the vibratory device 10 to provide a highly customizable experience. The processor 130 receives the feedback signal from the sensor 104 and creates a haptic signal based on this feedback and a manual signal from the manual actuator 92. This allows the device to adjust its vibrations in real-time in response to the user's movements and preferences.

The vibratory device 10 can also communicate with other devices via an optional wireless transceiver 140. This allows the user to control the device using a smartphone, tablet, or other smart device. The user can adjust the device's settings, switch between different vibration modes, and even program custom vibration patterns. This level of control allows the user to tailor the device's vibrations to their exact preferences, enhancing their pleasure and satisfaction. The vibratory device 10 is not only designed for pleasure but also for ease of use. The handle 90, manual actuator 92, and optional display make the device easy to control, even in the heat of the moment. The optional sleeve 100 and sensor 104 allow the device to respond to the user's movements, creating a more interactive and satisfying experience. And the wireless transceiver 140 allows the user to control the device using a familiar and intuitive interface: their own smartphone or tablet. That is, the vibratory device 10 is a versatile and customizable pleasure device. Its design combines comfort, control, and customization, allowing each user to tailor the device to their exact preferences. Whether used alone or with a partner, the vibratory device 10 is designed to enhance pleasure and satisfaction.

Referring now to FIG. 7, an alternate embodiment of the vibratory device 10 is depicted. Within the pocket portion 106, electronic components 108, including the haptic actuator 110, are positioned. More specifically, the electronic components include a controller 320 with driver functionality that is communicatively connected with the haptic actuator 110. In terms of driver functionality, the controller 320 may be a haptic driver that can operate a concentric rotating motor, or linear resonant actuator (LRA), or eccentric rotating mass (ERM) vibration motor, for example. The controller 320 in conjunction with the haptic actuator 110 may reduce the amperage requirements of the vibratory device 10 to extend battery life. A power source/battery 324 provides power to the electronic components. As shown, communication lines 326, 328 connect the controller to the actuatable members 102 and the sensor 104. Additionally, communication lines 330, 332 connect the haptic actuator 110 to the actuatable members 102 and the sensor 104. It should be appreciated that although a particular positioning of communication lines 326, 328, 330, 332 is depicted, the number and positioning of the communication lines 326, 328, 330, 332 may vary with the architectural requirements of the vibratory device 10, or a component thereof, such as the haptic actuator 110.

In one embodiment, the controller 320 receives various signals from the sensors 104. The controller 320 may then provide a haptic signal to the haptic actuator 110, which may produce different vibrations, which may be tapping sensations, in response to receiving different haptic signals. The vibratory device 10, as depicted in FIG. 7, is designed with user comfort and convenience in mind. The elongated shaft 80, with its rounded head portion 88, is ergonomically designed to fit comfortably within the pelvic cavity 12. The handle 90 at the base end 84 not only facilitates easy insertion and removal of the device but also houses the manual actuator 92, allowing the user to easily adjust the intensity and pattern of the vibrations.

The controller 320, which is part of the electronic components 108 housed within the pocket portion 106, plays a crucial role in the operation of the device. It receives signals from the sensor 104, which is designed to detect external pressure forces. These signals are then processed by the controller 320 to generate a haptic signal that is sent to the haptic actuator 110. The haptic actuator 110, in turn, generates vibrations that are transmitted to the actuatable members 102. This process allows the device to respond dynamically to the user's body, creating a highly personalized and interactive experience.

The vibratory device 10 also includes a power source/rechargeable battery 324, ensuring that the device can be used without the need for a constant power supply. This makes the device portable and convenient for use in various settings. The communication lines 326, 328, 330, and 332, which connect the various components, are strategically positioned to ensure efficient communication between the components while maintaining the compact design of the device. The vibratory device 10, therefore, represents a significant advancement in the field of personal pleasure devices. Its innovative design and advanced technology combine to create a device that not only enhances pleasure but also provides valuable feedback to the user, contributing to a better understanding of their body and their responses. This makes the vibratory device 10 not just a tool for pleasure, but also a valuable aid in promoting sexual health and wellness.

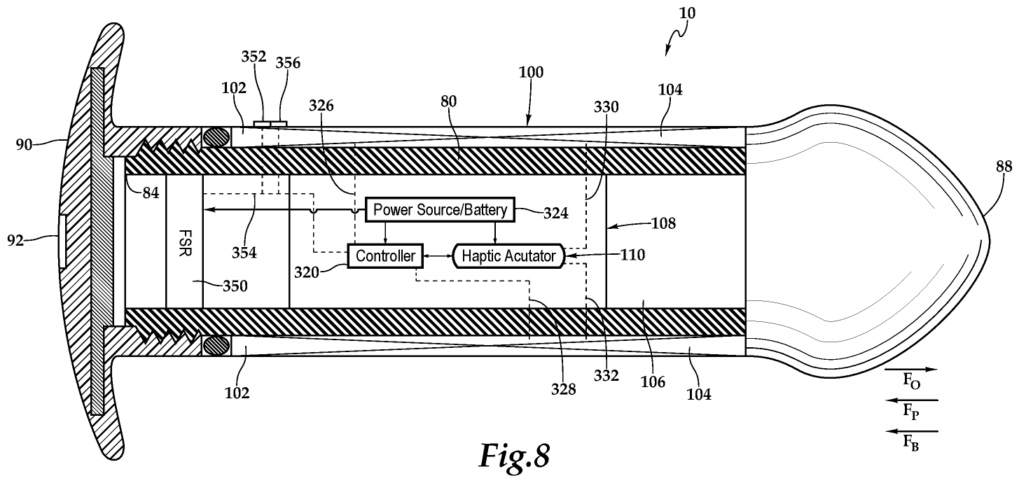

FIG. 8 illustrates an alternative embodiment of the vibratory device 10. The pocket portion 106 houses the electronic components 108, including the haptic actuator 110. Among these electronic components is a controller 320, equipped with driver functionality, which communicates with the haptic actuator 110. The controller 320 can function as a haptic driver, capable of operating various types of vibration motors, such as a concentric rotating motor, a linear resonant actuator (LRA), or an eccentric rotating mass (ERM) vibration motor. By working in tandem with the haptic actuator 110, the controller 320 can reduce the amperage requirements of the vibratory device 10, thereby extending the battery life. A power source/battery 324 supplies power to the electronic components. Communication lines 326 and 328 connect the controller to the actuatable members 102 and the sensor 104, while communication lines 330 and 332 link the haptic actuator 110 to the actuatable members 102 and the sensor 104. It is important to note that the depicted arrangement of communication lines 326, 328, 330, and 332 is just one possible configuration, and the number and placement of these lines can be adjusted to suit the architectural needs of the vibratory device 10 or its components, such as the haptic actuator 110.

In this embodiment, the vibratory device 10 is further equipped with a frequency adjustment sensor 350 and a manual intensity button 352. As shown communication lines 354 support communication between the frequency adjustment sensor 350, the manual intensity button 352, and the controller 320. The power source/battery 324 powers the frequency adjustment sensor 350, which communicates with the controller 320. The frequency adjustment sensor 350 allows users to modify the frequency of the vibrations produced by the haptic actuator 110, offering a more personalized and tailored sensory experience. The manual intensity button 352, also in communication with the controller 320, enables users to manually adjust the intensity or amplitude of the vibrations for a more comfortable experience.

In one embodiment, the controller 320 receives signals from both the sensors 104 and the frequency adjustment sensor 350. Based on these signals, the controller 320 generates a haptic signal that is sent to the haptic actuator 110, which can produce various vibrations, including tapping sensations, in response to different haptic signals. The vibratory device 10, as shown in FIG. 8, prioritizes user comfort and convenience. The elongated shaft 80 features a rounded head portion 88, ergonomically designed to fit comfortably within the pelvic cavity 12. The handle 90 at the base end 84 facilitates easy insertion and removal of the device and houses both the manual actuator 92 and the manual intensity button 352, allowing users to easily adjust the intensity and pattern of the vibrations. The manual intensity button 352 may serve as an intensity adjustment mechanism separate and distinct from the frequency adjustment sensor, allowing the user to adjust the intensity of the vibrations generated by the haptic actuator 110 through user. Also, a control element 356 may be used to adjust at least one of frequency and intensity of the vibrations generated by the haptic actuator 110.

The controller 320, a key component of the electronic components 108 located within the pocket portion 106, plays a vital role in the device's operation. It receives signals from the sensor 104, designed to detect external pressure forces, and the frequency adjustment sensor 350. The controller 320 processes these signals to produce a haptic signal that is transmitted to the haptic actuator 110. The haptic actuator 110 then generates vibrations that are conveyed to the actuatable members 102, enabling the device to dynamically respond to the user's body for a highly personalized and interactive experience.

The vibratory device 10 also features a power source/rechargeable battery 324, allowing for use without a constant power supply and making the device portable and convenient for various settings. The communication lines 326, 328, 330, 332, and 354 connecting the different components, are strategically arranged to ensure efficient communication while maintaining the device's compact design. The vibratory device 10 represents a significant advancement in personal pleasure devices, combining innovative design and advanced technology to enhance pleasure and provide valuable feedback to users, contributing to a better understanding of their bodies and responses. As such, the vibratory device 10 serves not only as a tool for pleasure but also as a valuable resource for promoting sexual health and wellness.

The order of execution or performance of the methods and data flows illustrated and described herein is not essential, unless otherwise specified. That is, elements of the methods and data flows may be performed in any order, unless otherwise specified, and that the methods may include more or less elements than those disclosed herein. For example, it is contemplated that executing or performing a particular element before, contemporaneously with, or after another element are all possible sequences of execution.

While this invention has been described with reference to illustrative embodiments, this description is not intended to be construed in a limiting sense. Various modifications and combinations of the illustrative embodiments as well as other embodiments of the invention, will be apparent to persons skilled in the art upon reference to the description. It is, therefore, intended that the appended claims encompass any such modifications or embodiments.

1. A vibratory device for pleasure enhancement, the vibratory device comprising:
an elongated shaft having an insertion end and a base end with an external surface portion therebetween, the elongated shaft designed for insertion within a pelvic cavity, the elongated shaft housing a processor, memory, and storage communicatively interconnected in a busing architecture;
a manual actuator communicatively interconnected to the busing architecture, the manual actuator generating a manual signal in response to manual actuation thereof; and
a haptic actuator communicatively interconnected to the busing architecture, the haptic actuator having processing that separates amplitude from frequency, the haptic actuator, in response to receiving a haptic signal, generating a plurality of vibrations via actuatable elements integrated into the elongated shaft.

2. The vibratory device as recited in claim 1, wherein the haptic actuator further comprises a vibration frequency range of 0 Hz to 120 Hz.

3. The vibratory device as recited in claim 1, wherein the haptic actuator further comprises a manual tapping output mode.

4. The vibratory device as recited in claim 1, wherein the haptic actuator further comprises a magnetic wideband vibrational actuator.

5. The vibratory device as recited in claim 1, wherein the haptic actuator further comprises a magnet and a hollow member, including a voice coil therein, have relative longitudinal movement therebetween with thin elastic membranes interconnecting the magnet and the hollow member.

6. The vibratory device as recited in claim 1, wherein the haptic actuator further comprises an actuator selected from the group consisting of a magnetic wideband vibrational actuator, a solenoid actuator vibrator, a linear resonant actuator vibration motor, and a Linear Magnetic Ram (LMR) actuator.

7. A vibratory device for pleasure enhancement, the vibratory device comprising:
an elongated shaft having an insertion end and a base end with an external surface portion therebetween, the elongated shaft designed for insertion within a pelvic cavity, the elongated shaft housing a processor, memory, and storage communicatively interconnected in a busing architecture;
a manual actuator communicatively interconnected to the busing architecture, the manual actuator generating a manual signal in response to manual actuation thereof;
a haptic actuator communicatively interconnected to the busing architecture, the haptic actuator having processing that separates amplitude from frequency, the haptic actuator, in response to receiving a haptic signal, generating a plurality of vibrations via actuatable elements integrated into the elongated shaft; and
the memory being accessible to the processor, the memory including processor-executable instructions that, when executed by the processor, cause the processor to:
create the haptic signal based on the manual signal and the feedback signal, and
drive the haptic signal to the haptic actuator.

8. The vibratory device as recited in claim 7, wherein the haptic actuator further comprises a vibration frequency range of 0 Hz to 120 Hz.

9. The vibratory device as recited in claim 7, wherein the haptic actuator further comprises a manual tapping output mode.

10. The vibratory device as recited in claim 7, wherein the haptic actuator further comprises a magnetic wideband vibrational actuator.

11. The vibratory device as recited in claim 7, wherein the haptic actuator further comprises a magnet and a hollow member, including a voice coil therein, have relative longitudinal movement therebetween with thin elastic membranes interconnecting the magnet and the hollow member.

12. The vibratory device as recited in claim 7, wherein the haptic actuator further comprises an actuator selected from the group consisting of a magnetic wideband vibrational actuator, a solenoid actuator vibrator, a linear resonant actuator vibration motor, and a Linear Magnetic Ram (LMR) actuator.

13. The vibratory device as recited in claim 7, wherein the haptic actuator comprises access to a waveform memory.

14. The vibratory device as recited in claim 7, wherein the haptic signal further comprises an amplifier-modulated haptic signal.

15. The vibratory device as recited in claim 7, further comprising an amplifier interposed between the haptic actuator and the busing architecture.

16. The vibratory device as recited in claim 7, wherein the haptic actuator is at least partially integrated into the elongated shaft.

17. A vibratory device for pleasure enhancement, the vibratory device comprising:
an elongated shaft having an insertion end and a base end with an external surface portion therebetween, the elongated shaft designed for insertion within a pelvic cavity, the elongated shaft housing a processor, memory, and storage communicatively interconnected in a busing architecture;
a manual actuator communicatively interconnected to the busing architecture, the manual actuator generating a manual signal in response to manual actuation thereof;
a haptic actuator communicatively interconnected to the busing architecture, the haptic actuator having processing that separates amplitude from frequency, the haptic actuator, in response to receiving a haptic signal, generating a plurality of vibrations via actuatable elements integrated into the elongated shaft; and
the memory being accessible to the processor, the memory including processor-executable instructions that, when executed by the processor, cause the processor to:
drive the haptic signal to the haptic actuator.

18. The vibratory device as recited in claim 17, wherein the haptic actuator further comprises a vibration frequency range of 0 Hz to 80 Hz.

19. The vibratory device as recited in claim 17, wherein the haptic actuator further comprises an actuator selected from the group consisting of a magnetic wideband vibrational actuator, a solenoid actuator vibrator, a linear resonant actuator vibration motor, and a Linear Magnetic Ram (LMR).

20. The vibratory device as recited in claim 17, wherein the haptic signal further comprises an amplifier-modulated haptic signal.

21. A vibratory device for pleasure enhancement, the vibratory device comprising:
an elongated shaft having an insertion end and a base end with an external surface portion therebetween, the elongated shaft designed for insertion within a pelvic cavity, the elongated shaft housing a processor, memory, and storage communicatively interconnected in a busing architecture;
a manual actuator communicatively interconnected to the busing architecture, the manual actuator generating a manual signal in response to manual actuation thereof;
a haptic actuator communicatively interconnected to the busing architecture, the haptic actuator having processing that separates amplitude from frequency, the haptic actuator, in response to receiving a haptic signal, generating a plurality of vibrations via actuatable elements integrated into the elongated shaft;
a frequency adjustment sensor communicatively interconnected to the busing architecture, the frequency adjustment sensor generating a frequency adjustment signal in response to user interaction, the processor receiving the frequency adjustment signal and adjusting the frequency of the vibrations generated by the haptic actuator based on the frequency adjustment signal;
an intensity adjustment mechanism separate and distinct from the frequency adjustment sensor, allowing the user to adjust the intensity of the vibrations generated by the haptic actuator through user interaction; and
a control element being used to adjust at least one of frequency and intensity of the vibrations generated by the haptic actuator.
